(19) United States
(12) Patent Application Publication
(10) Pub. No.: US 2026/0182823 A1
(43) Pub. Date: Jul. 2, 2026

(54) METHOD, COMPUTING DEVICE AND COMPUTER PROGRAM FOR ANALYZING ENDOSCOPIC VIDEO

(71) Applicant: MedInTech Inc., Seoul (KR)

(72) Inventors: Chiwon LEE, Namyangju (KR); Myungjoon Kim, Gwacheon (KR); Seokho Cho, Seoul (KR)

(21) Appl. No.: 19/394,992

(22) Filed: Nov. 20, 2025

(30) Foreign Application Priority Data
Dec. 30, 2024 (KR) 10-2024-0200542

(51) Int. Cl. A61B 1/00; G06T 7/00; G06V 20/40

(52) U.S. Cl. CPC A61B 1/00009; G06T 7/0012; G06V 20/41

(57) ABSTRACT

According to an embodiment of the present invention, there is disclosed a method of analyzing endoscopic video that is performed by a computing device including at least one processor. The method includes: determining a frame-wise class corresponding to an operation to be performed by an endoscopic device for each frame in an endoscopic video including a plurality of frames; and determining a final class corresponding to a final operation to be performed by the endoscopic device for the endoscopic video based on the frame-wise class. The class may comprise at least one class selected from the group consisting of a first class for operating the air pump of the endoscopic device, a second class for operating the water pump of the endoscopic device, a third class for operating the suction pump of the endoscopic device, and a fourth class corresponding to cases other than cases of the first to third classes.

### CROSS-REFERENCE TO RELATED APPLICATION

This application claims the benefit of Korean Patent Application No. 10-2024-0200542 filed on December 30, 2024, which is hereby incorporated by reference herein in its entirety.

### BACKGROUND

### 1. Technical Field

The present disclosure relates to technology for analyzing endoscopic video, and more particularly, to a method, computing device, and computer program for analyzing endoscopic video that determine a final operation to be performed by an endoscopic device based on an endoscopic video.

### 2. Description of the Related Art

An endoscope is a general name for a medical device that allows a scope to be inserted into the body of a human or animal and the inside of the body to be observed without surgery or an autopsy. An endoscope inserts a scope into the inside of the body, radiates light, and visualizes the light reflected from the surface of an internal wall. Such endoscopes are categorized by purpose and body part. They may be basically categorized into rigid endoscopes, which have metal tubes, and flexible endoscopes, which are represented by digestive endoscopes.

Meanwhile, during endoscopic procedures, medical staff use various pumps provided in an endoscopic device in order to secure a clear field of view and perform a safe procedure. As an example, an air pump can be used to inject air in order inflate a digestive organ to facilitate the passage of a scope or in order to smooth out wrinkles within the organ to secure a clear field of view. A water pump can also be used to inject water during an endoscopic procedure in order to clean the inside of an organ or the lens of the scope or remove obstructions to maintain a clear field of view. Additionally, a water pump can be used to hydrate tissue in order to facilitate incision or biopsy in a specific procedure. A suction pump can also be used to suction foreign substances in order to suction blood, mucus, and/or water generated during an endoscopic procedure, in order to enhance the safety of an endoscopic procedure, and in order to prevent post-procedure complications.

These pumps are frequently used during endoscopic procedures, and thus medical staff inevitably suffers from cumulative fatigue by repeatedly performing these operations during the procedures. Accordingly, there is a demand for technology that assists the operation of endoscopic pumps. Additionally, given the nature of endoscopic procedures in which medical staff needs to perform endoscopic procedures in real time while viewing endoscopic video, the technology that assists the operation of the endoscopic pumps needs to provide stable and consistent results.

### SUMMARY

The present disclosure is intended to overcome the above-described problems of the related art, and is directed to a method, computing device, and computer program for analyzing endoscopic vide that determine a final operation to be performed by an endoscopic device based on an endoscopic video.

However, objects to be accomplished by the present disclosure are not limited to the object described above, and there may be other objects.

According to an aspect of the present invention, there is provided a method of analyzing endoscopic video that is performed by a computing device. The method includes: determining a frame-wise class corresponding to an operation to be performed by an endoscopic device for each frame in an endoscopic video including a plurality of frames; and determining a final class corresponding to a final operation to be performed by the endoscopic device for the endoscopic video based on the frame-wise class. The class may comprise at least one class selected from the group consisting of a first class for operating the air pump of the endoscopic device, a second class for operating the water pump of the endoscopic device, a third class for operating the suction pump of the endoscopic device, and a fourth class corresponding to cases other than cases of the first to third classes.

Alternatively, determining the final class may include determining the final class based on a class determined for at least one previous frame earlier than a first frame or a class determined for at least one subsequent frame later than the first frame.

Alternatively, determining the final class may include determining the final class based on the type and number of frame-wise classes determined for the plurality of frames.

Alternatively, determining the frame-wise class may include storing the class determined for the at least one previous frame before the class determined for the first frame, and storing the class determined for the at least one subsequent frame after the class determined for the first frame.

Alternatively, determining the final class may include: when the first class is stored successively for a first number of times, determining the final class to be the first class; when the second class is stored successively for a second number of times, determining the final class to be the second class; and, when the third class is stored successively for a third number of times, determining the final class to be the third class.

Alternatively, the first number or the second number may be larger than the third number.

Alternatively, the method may further include controlling at least one of the air pump, the water pump, or the suction pump to perform an operation corresponding to the final class.

Alternatively, determining the frame-wise class may include determining an image containing secretions inside a body, into which a scope of the endoscopic device is inserted, to be the third class.

Alternatively, determining the frame-wise class may include determining an image, corresponding to a case where the scope of the endoscopic device comes into contact with the inner wall of the body, to be the fourth class.

Alternatively, determining the frame-wise class may include determining an image, containing noise caused by the movement of a scope, to be the fourth class.

Alternatively, the noise may include a defocused image or motion blur.

According to another aspect of the present invention, there is provided a computing device for analyzing endoscopic video. The computing device includes: memory configured to store an endoscopic video including a plurality of frames; and a processor configured to determine a frame-wise class corresponding to an operation to be performed by an endoscopic device for each of the frames and determine a final class corresponding to a final operation to be performed by the endoscopic device for the endoscopic video based on the frame-wise class. The class includes at least one class selected from the group consisting of a first class for operating an air pump of the endoscopic device, a second class for operating a water pump of the endoscopic device, a third class for operating a suction pump of the endoscopic device, and a fourth class corresponding to cases other than cases of the first to third classes.

According to another aspect of the present invention, there is provided a computer program stored in a computer-readable storage medium. The computer program causes operations for analyzing endoscopic video to be performed when executed by at least one processor. The operations include operations of: determining a frame-wise class corresponding to an operation to be performed by an endoscopic device for each frame in an endoscopic video including a plurality of frames; and determining a final class corresponding to a final operation to be performed by the endoscopic device for the endoscopic video based on the frame-wise class. The class comprises at least one class selected from the group consisting of a first class for operating an air pump of the endoscopic device, a second class for operating a water pump of the endoscopic device, a third class for operating a suction pump of the endoscopic device, or a fourth class corresponding to cases other than cases of the first to third classes.

According to at least one embodiment of the present disclosure, the pump of an endoscopic device is controlled using a result output from a single artificial neural network model, and thus it may be possible to prevent instability that may occur when models that individually determine each class are employed.

Furthermore, according to at least one embodiment of the present disclosure, an endoscopic device may be stably controlled by taking into consideration the similarity or consistency between classes determined on a frame-wise basis.

### BRIEF DESCRIPTION OF THE DRAWINGS

The above and other objects, features, and advantages of the present invention will be more clearly understood from the following detailed description taken in conjunction with the accompanying drawings, in which:

FIG. 1 is a block diagram of an endoscopic system including an endoscopic device and a computing device according to one embodiment of the present disclosure;

FIG. 2 is a diagram showing the configuration of an endoscopic device according to one embodiment of the present disclosure;

FIG. 3 is an exemplary diagram showing an artificial neural network model according to one embodiment of the present disclosure;

FIGS. 4A, 4B, and 4C are exemplary diagrams showing a method of operating a computing device according to one embodiment of the present disclosure; and

FIG. 5 is a flowchart showing a method by which a computing device controls an endoscopic device according to one embodiment of the present disclosure.

### DETAILED DESCRIPTION

Embodiments of the present disclosure will be described in detail below with reference to the accompanying drawings so that those having ordinary skill in the art of the present disclosure (hereinafter referred to as those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Additionally, in order to clearly describe the present disclosure, reference numerals for parts that are not related to the description of the present disclosure may be omitted in the drawings.

The term "or" used herein is intended not to mean an exclusive "or" but to mean an inclusive "or." That is, unless otherwise specified herein or the meaning is not clear from the context, the clause "X uses A or B" should be understood to mean one of the natural inclusive substitutions. For example, unless otherwise specified herein or the meaning is not clear from the context, the clause "X uses A or B" may be interpreted as any one of a case where X uses A, a case where X uses B, and a case where X uses both A and B.

The term "and/or" used herein should be understood to refer to and include all possible combinations of one or more of listed related concepts.

The terms "include" and/or "including" used herein should be understood to mean that specific features and/or components are present. However, the terms "include" and/or "including" should be understood as not excluding the presence or addition of one or more other features, one or more other components, and/or combinations thereof.

Unless otherwise specified herein or unless the context clearly indicates a singular form, the singular form should generally be construed to include "one or more."

The term "N-th (N is a natural number)" used herein can be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

Meanwhile, the term "module" or "unit" used herein may be understood as a term referring to an independent functional unit processing computing resources, such as a computer-related entity, firmware, software or part thereof, hardware or part thereof, or a combination of software and hardware. In this case, the "module" or "unit" may be a unit composed of a single component, or may be a unit expressed as a combination or set of multiple components. For example, in the narrow sense, the term "module" or "unit" may refer to a hardware component or set of components of a computing device, an application program performing a specific function of software, a procedure implemented through the execution of software, a set of instructions for the execution of a program, or the like. Additionally, in the broad sense, the term "module" or "unit" may refer to a computing device itself constituting part of a system, an application running on the computing device, or the like. However, the above-described concepts are only examples, and the concept of "module" or "unit" may be defined in various manners within a range understandable to those skilled in the art based on the content of the present disclosure.

The term "model" used herein may be understood as a system implemented using mathematical concepts and language to solve a specific problem, a set of software units intended to solve a specific problem, or an abstract model for a process intended to solve a specific problem. For example, a neural network "model" may refer to an overall system implemented as a neural network that is provided with problem-solving capabilities through training. In this case, the neural network may be provided with problem-solving capabilities by optimizing parameters connecting nodes or neurons through training. The neural network "model" may include a single neural network, or a neural network set in which multiple neural networks are combined together.

The term "image" used herein may refer to multidimensional data composed of discrete image elements. In other words, the term "image" may be understood as a term that refers to a digital representation of an object visible to the human eye. For example, the term "image" may refer to multidimensional data composed of elements corresponding to pixels in a two-dimensional image. Alternatively, the term "image" may refer to multidimensional data composed of elements corresponding to voxels in a three-dimensional image.

The term "video" used herein may refer to multidimensional data composed of a plurality of temporally consecutive "images." In other words, the term "video" may be understood as a term that refers to a digital representation that changes over time. For example, the term "video" may refer to data composed of a plurality of two-dimensional images (frames) captured at regular time intervals, and each of these frames may be multidimensional data composed of pixels. Additionally, the "video" may contain data generated by sequentially displaying three-dimensional images along a time axis. In this case, each frame may be multidimensional data composed of voxels.

The foregoing descriptions of the terms are intended to help to understand the present disclosure. Accordingly, it should be noted that unless the above-described terms are explicitly described as limiting the content of the present disclosure, the terms in the content of the present disclosure are not used in the sense of limiting the technical spirit of the present disclosure.

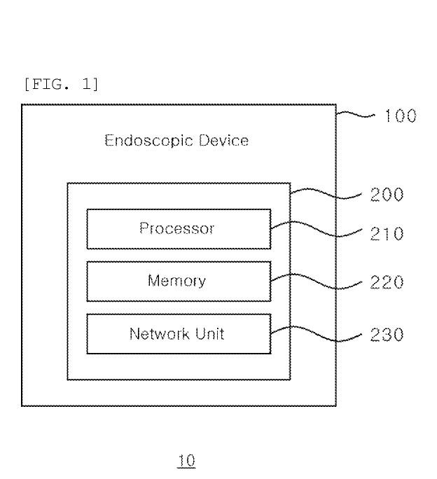

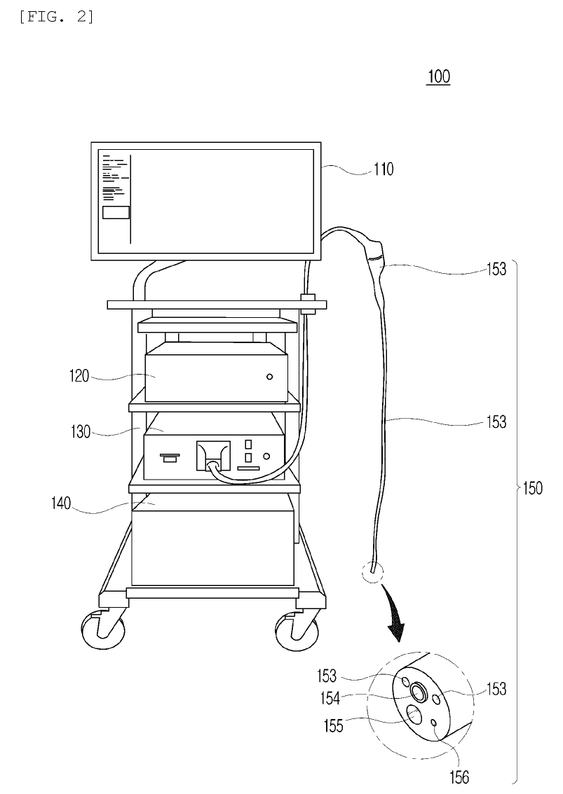

FIG. 1 is a block diagram of an endoscopic system including an endoscopic device and a computing device according to one embodiment of the present disclosure, and FIG. 2 is a diagram showing the configuration of an endoscopic device according to one embodiment of the present disclosure.

Referring to FIG. 1, an endoscope system 10 includes an endoscopic device 100 configured to obtain various types of information including medical images of the inside of the body, transmit the medical images to a computing device 200 and receive control signals from the computing device 200, and the computing device 200 configured to acquire medical images via the endoscopic device 100 and generate signals for controlling the endoscopic device 100 based on the medical images.

In the present specification, the endoscopic device 100 may be a rigid endoscope or a flexible endoscope. The rigid endoscope may include a straight tube made of high-strength metal or plastic, and the flexible endoscope may include a tube made of flexible material. Such a tube may be referred to as a scope 150. The scope 150 of each of the rigid and flexible endoscopes may include an optical fiber or lens system for transmitting light and images therein. Furthermore, a channel for spraying air or water, a channel for inserting a biopsy instrument, and/or the like may be provided. The operations and configurations described in the present disclosure may be intended to control a rigid endoscope or a flexible endoscope. However, the type of endoscopic device 100 to which the present disclosure is applied is not limited thereto.

Furthermore, in the present specification, the endoscopic device 100 may be used for an animal or a human. That is, the "body" described herein may refer to either the body of an animal or the body of a human. The configuration of the endoscopic device 100 may be appropriately modified by taking into consideration the size of the body of an animal or a human and anatomical characteristics, and the operations and configurations described herein may also be appropriately modified according to the characteristics of the body.

The endoscopic device 100 may include a component capable of acquiring a medical image of the inside of the body, and a component capable of, if necessary, inserting an instrument to perform treatment or a procedure while viewing a medical image. The endoscopic device 100 may include a control unit 120 configured to control the overall operation of the endoscopic device 100, a scope 150 configured to be inserted into the body, and a driving unit 130 configured to provide the power required for the movement of the scope 150.

Referring to FIG. 2, the endoscopic device 100 may include an output unit 110, the control unit 120, the driving unit 130, a pump unit 140, and the scope 150, and may further include a light source unit (not shown).

The output unit 110 may include a display that displays medical images. The output unit 110 may include a display module capable of outputting visualized information or implementing a touch screen, such as a liquid crystal display (LCD), a thin film transistor-liquid crystal display (TFT LCD), an organic light-emitting diode (OLED) display, a flexible display, or a three-dimensional (3D) display.

The output unit 110 may include various means for providing medical images or information about medical images. The output unit 110 may display medical images acquired via the scope 150 without changes, or may display medical images processed by the control unit 120. Alternatively, the output unit 110 may output information, received via the computing device 200, to the outside.

The output unit 110 may provide information through auditory means other than visual means. For example, a speaker configured to provide audible alarms for medical images. Meanwhile, although the single output unit 110 is shown in FIG. 2, there may be a plurality of output units 110. In this case, some of the output units may display medical images acquired via the scope 150, while others may display information processed by the control unit 120 or the computing device 200.

The control unit 120 may control the overall operation of the endoscopic device 100. For example, the control unit 120 may perform the operation of capturing a medical image via the scope, the operation of processing an acquired medical image, a control operation for performing a medical operation such as the spraying of cleaning water or suctioning, and a series of operations for controlling the movement of the scope.

In the present specification, the control unit 120 may process information obtained via the endoscopic device 100 for provision to the computing device 200. The control unit 120 may process information received from the computing device 200 to generate a signal for controlling the endoscopic device 100. As an example, the control unit 120 may generate a control signal for operating at least one of the air pump, water pump, or suction pump of the endoscopic device 100 based on the class determination result generated by the computing device 200.

The control unit 120 may include any type of device capable of processing data. According to an exemplary embodiment, the control unit 120 may be a hardware-embedded data processing device having a physically structured circuit to perform functions expressed in code or instructions included in a program. Examples of the hardware-embedded data processing device may include processing devices such as a microprocessor, a central processing unit (CPU), a processor core, a multiprocessor, an application-specific integrated circuit (ASIC), or a field programmable gate array (FPGA), but the technical concept of the present disclosure is not limited thereto.

The control unit 120 may control the movement of the scope 150 through the driving unit 130 connected to the scope 150. That is, the control unit 120 may generate a control signal to be provided to the driving unit 130 in order to control the movement of the scope 150. For example, a series of operations by which the endoscopic device 100 of the present disclosure controls the scope 150 may be performed as follows. The user may input the degree or direction of curvature of the scope 150 through a manipulation unit 151. The input information may be transmitted to the control unit 120, and the control unit 120 may then process the input information and generate a signal to be provided to the driving unit 130. For example, the control unit 120 may calculate the position, angle, angular velocity, and/or the like of a motor corresponding to the degree or direction of curvature set by a user, and may provide the results of the calculation to the driving unit 130. The driving unit 130 may generate power based on the signal from the control unit 120 and transmit this power to the scope 150. Accordingly, the scope 150 may be moved or bent in response to the values input by the user.

The driving unit 130 may provide the power required for the scope to be inserted into the body, to be bent within the body, and to be moved within the body. For example, the driving unit 130 may include motors connected to wires within the scope, and a tension adjustment unit configured to adjust the tension of the wires.

The driving unit 130 may control the scope in various directions by controlling the power of the motors. For example, the motors may include a plurality of motors that correspond to the directions in which an insertion portion 152 at an end of the scope 150 needs to be bent. Alternatively, the motors may include a plurality of motors that correspond to wires within the scope 150. More specifically, the driving unit 130 may include a first motor configured to determine the x-axis movement of the scope 150, and a second motor configured to determine the y-axis movement of the scope 150. The x-axis location, y-axis location, z-axis location, and roll, pitch, and yaw values of the end of the scope 150 may be determined based on the control of the driving unit 130, but the configuration of the driving unit 130 is not limited thereto.

The tension adjustment unit may generate tension by receiving power from the motors and pulling the wires inside the scope 150. This allows the scope 150 to be bent. The tension adjustment unit may adjust the tension applied to the plurality of wires inside the scope 150 so that the scope 150 can be bent according to the determined amount and direction of curvature.

The pump unit 140 may include at least one of an air pump configured to inject air into the body through the scope 150, a suction pump configured to provide negative pressure or vacuum to suck at least one of gas, liquid, or a foreign substance from the inside of the body through the scope 150, or a water pump configured to inject cleaning water into the body through the scope 150. Each of the pumps may include a valve for controlling the flow of fluid. The pump unit 140 may be selectively opened and closed by the control unit 120. At least one of the suction pump, the water pump, or the air pump may be selectively opened and closed based on a control signal from the computing device 200 or the control of the control unit 120.

The scope 150 may include an insertion part 152 configured to be inserted into a digestive organ, and a manipulation part 151 configured to control the movement of the insertion part 152 and receive input from the user to perform various operations.

The insertion part 152 is configured to be flexible, and is connected to the driving unit 130 at one end thereof, so that the degree and direction of curvature can be determined by the driving unit 130. Since medical imaging and procedures are performed at the end of the insertion part 152, the scope 150 may include pluralities of cables and tubes that extend to the end of the insertion part. The scope 150 may include a light source lens 153, an objective lens 154, a working channel 155, and an air/water channel 156. An instrument for treating or manipulating a lesion during an endoscopic procedure may be inserted through the working channel. Air may be injected and cleaning water may be supplied through the air/water channel. Meanwhile, although the air/water channel is shown as the channel through which cleaning water is supplied in FIG. 2, it is not limited thereto. As an example, a separate water jet channel (not shown) may be provided within the scope 150, and cleaning water may also be supplied through the water jet channel.

Meanwhile, the expression "the scope is bent by the control unit 120 or driving unit 130" described herein may refer to a case where at least a portion, such as the insertion part 152, of the scope 150 is bent.

The manipulation unit 151 may include a plurality of input buttons that allow an endoscopist to control the steering of the insertion part 152 and provide various functions (such as the capturing of an image, and the spraying of cleaning water) to perform procedures through the working channel 155 and the air/water channel 156. For example, the manipulation unit 151 may include a plurality of buttons or a joystick-type input device that indicates the direction of the scope 150.

The light source unit may include a light source that radiates light into the body through the endoscopic scope 150. The light source unit may include a lighting device that generates white light, or may include a plurality of lighting devices that generate light with different wavelength bands. The type of light source, the intensity of light, white balance, and/or the like may be set through the light source unit. Meanwhile, the above-described settings may also be set through the control unit 120. The light generated by the light source unit may be transmitted to the scope 150 through a path such as an optical fiber.

The computing device 200 may receive information from the endoscopic device 100, and, based on the received information, generate a signal for controlling the endoscopic device 100 and provide the signal to the endoscopic device 100.

Meanwhile, although the computing device 200 is shown as being located within the endoscopic device 100 in FIG. 1, the computing device 200 may be provided outside the endoscopic device 100. In this case, the endoscopic device 100 and the computing device 200 may be connected over a wired/wireless network, and may transmit and receive data. Furthermore, the endoscopic system 10 may be operated in a cloud environment. In this case, the endoscopic system 10 may further include the endoscopic device 100, the computing device 200, and a cloud server.

The computing device 200 according to one embodiment of the present disclosure may be a hardware device that performs comprehensive data processing and computational operations or a part of the hardware device, or may be a software-based computing environment that is connected over a communication network. For example, the computing device 200 may be a server that is a subject that performs intensive data processing functions and shares resources, or may be a client that shares resources through interaction with a server. Furthermore, the computing device 200 may be a cloud system that enables pluralities of servers and clients to interact and comprehensively process data. The above description is merely one example of the type of computing device 200. Accordingly, the type of computing device 200 may vary within a range understandable to those skilled in the art based on the content of the present disclosure. The above description is merely one example of the type of computing device 200. Accordingly, the type of computing device 200 may be configured in various manners within a range understandable to those skilled in the art based on the content of the present disclosure.

Referring to FIG. 1, the computing device 200 according to one embodiment of the present disclosure may include a processor 210, memory 220, and a network unit 230. However, FIG. 1 is merely an example, and the computing device 200 may include other components for implementing a computing environment. Furthermore, only some of the components disclosed above may be included in the computing device 200.

The processor 210 according to one embodiment of the present disclosure may be understood as a constituent unit including hardware and/or software for performing computing operations. For example, the processor 210 may read a computer program and perform data processing for machine learning. The processor 210 may process operation processes such as the processing of input data for machine learning, the extraction of features for machine learning, and the computation of errors based on backpropagation. The processor 210 for performing such data processing may include a central processing unit (CPU), a general purpose graphics processing unit (GPGPU), a tensor processing unit (TPU), an application specific integrated circuit (ASIC), and a field programmable gate array (FPGA). Since the types of processor 210 described above are only examples, the type of processor 210 may be configured in various manners within a range understandable to those skilled in the art based on the content of the present disclosure.

According to the present disclosure, the processor 210 may generate a signal for controlling the endoscopic device 100 based on an endoscopic image of the inside of the body acquired via the endoscopic device 100. The signal may be a signal for selectively operating a pump of the endoscopic device 100 and stopping the operation of the pump. The processor 210 may generate a signal for selectively operating at least one of an air pump, a water pump, or a suction pump and stopping the operation thereof based on the endoscopic image. For this operation, the processor 210 may train an artificial neural network model 300 and utilize the trained artificial neural network model 300.

In the following, in the present specification, one of a series of endoscopic images successively acquired from the endoscopic device 100 is referred to as a frame. A group of endoscopic images corresponding to the type of operation of the endoscopic device 100 is referred to as a class. As an example, a first class may refer to a group of endoscopic images that require the operation of the air pump of the endoscopic device 100, a second class may refer to a group of endoscopic images that require the operation of the water pump of the endoscopic device 100, and a third class may refer to a group of endoscopic images that require the operation of the suction pump of the endoscopic device 100. A class that does not fall into the first to third classes may be referred to as a fourth class.

In the present disclosure, as an example, the classes of endoscopic images may be determined based on the pump type of the endoscopic device 100. However, the above criterion is exemplary, and the class determination criterion may vary depending on the controllable component of the endoscopic device. That is, classes may be determined based on the component to be operated in the endoscopic device.

The processor 210 may train the artificial neural network model 300 that determines which of the various pumps of the endoscopic device 100 needs to be operated based on an endoscopic image. The endoscopic image may then be input to the trained artificial neural network model 300 to generate signals to operate and stop the pump. That is, the trained artificial neural network model 300 may determine a class corresponding to the endoscopic image and generate a class determination result.

The processor 210 may generate training data by labeling an endoscopic image as a first, second, third, or fourth class. The training data may then be used to train the artificial neural network model 300 to infer the operation to be performed by the endoscopic device 100 based on the endoscopic image.

The processor 210 may use the artificial neural network model 300 trained to infer the operation to be performed by the endoscopic device 100 based on the endoscopic image to determine a class corresponding to at least one endoscopic image acquired via the endoscopic device 100.

The processor 210 may determine the final operation to be performed by the endoscopic device 100 for an endoscopic video based on a class result for each frame. That is, the final class may be determined based on a frame-wise class. The endoscopic device 100 may be controlled based on the final class.

The processor 210 may continuously determine the class for each frame for continuously acquired frames. That is, control signals for controlling the endoscopic device 100 are not generated based on the class for any one frame. How continuous the class distribution is for respective frames in an endoscopic video is taken into consideration. This is intended to ensure the stable operation of the endoscopic device 100 by taking into consideration the similarity or consistency between the classes determined for respective frames.

Meanwhile, when a plurality of models are employed to individually determine each class unlike in the present disclosure, there may arise a situation in which a given endoscopic image is determined to belong to both the first and third classes. When the endoscopic device 100 is controlled using such a result, post-processing is necessarily required to ensure operational stability. This may reduce the operation speed of the endoscopic device 100, where real-time performance is critical. Accordingly, according to the present disclosure, the computing device 200 controls the pump of the endoscopic device 100 by using the results output from the single artificial neural network model 300. Accordingly, this prevents a situation where the pump is used repeatedly, which may occur when each class is individually determined.

The memory 220 according to one embodiment of the present disclosure may be understood as a constituent unit including hardware and/or software for storing and managing data that is processed in the computing device 200. That is, the memory 220 may store any type of data generated or determined by the processor 210 and any type of data received by the network unit 230. For example, the memory 220 may include at least one type of storage medium of a flash memory type, hard disk type, multimedia card micro type, and card type memory, random access memory (RAM), static random access memory (SRAM), read-only memory (ROM), electrically erasable programmable read-only memory (EEPROM), programmable read-only memory (PROM), magnetic memory, a magnetic disk, and an optical disk. Furthermore, the memory 220 may include a database system that controls and manages data in a predetermined system. Since the types of memory 220 described above are only examples, the type of memory 220 may be configured in various manners within a range understandable to those skilled in the art based on the content of the present disclosure.

The memory 220 may structure, organize, and manage the data required for the processor 210 to perform operations, data combinations, and program code executable by the processor 210. Furthermore, the memory 220 may store program codes that cause the processor 210 to generate training data.

The memory 220 may store an endoscopic video acquired via the endoscopic device 100. The memory 220 may also store parameters generated during a process in which the processor 210 trains the artificial neural network model 300.

The memory 220 may store information about individual frames constituting an endoscopic video used as training data and labels corresponding to the frames. For example, the memory 220 may store an image labeled as the first class, which is an image corresponding to a case where the operation of the air pump is required because the image is out of focus or a foreign substance is present the lens, requiring the cleaning of the lens of the scope 150, or a case where an observation area formed by the inner wall of a digestive organ is narrow. The memory 220 may store an image labeled as the second class, which is an image corresponding to a case where the operation of the water pump is required because water or a foreign substance is attached to both corners of the lens of the scope 150, requiring the cleaning of the lens, or a case where the operation of the water pump is required because a foreign substance or blood is present in a lesion region, requiring the cleaning of the affected area. The memory 220 may store an image labeled as the third class, which is an image corresponding to a case where the inside of the body, into which the scope 150 of the endoscopic device 100 is inserted, contains secretions, requiring the operation of the suction pump. The memory 220 may store an image labeled as the fourth class, which is an image corresponding to a case where the scope 150 of the endoscopic device 100 comes into contact with the inner wall of the body or which is an image containing the noise caused by the movement of the scope 150.

The network unit 230 according to one embodiment of the present disclosure may be understood as a constituent unit that transmits and receives data via any type of known wired/wireless communication system. For example, the network unit 230 may perform data transmission and reception by using a wired/wireless communication system such as a local area network (LAN), a wideband code division multiple access (WCDMA) network, a long term evolution (LTE) network, the wireless broadband Internet (WiBro), a 5th generation mobile communication (5G) network, an ultra-wideband wireless communication network, a ZigBee network, a radio frequency (RF) communication network, a wireless LAN, a wireless fidelity network, a near field communication (NFC) network, or a Bluetooth network. Since the above-described communication systems are only examples, the wired/wireless communication system for the data transmission and reception of the network unit 230 may be applied in various manners other than the above-described examples.

The network unit 230 may receive data, required for the processor 210 to perform operations, through wired/wireless communication with any system, any client, or the like. Furthermore, the network unit 230 may transmit data, generated through the operations of the processor 210, through wired/wireless communication with any system, any client, or the like. For example, the network unit 230 may receive data, including an endoscopic image, through communication with a picture archiving and communication system (PACS), a cloud server that performs tasks such as the standardization of medical data, the endoscopic device 100, or the like. The network unit 230 may transmit various types of data generated via the operations of the processor 210 through communication with the above-described system, server, or endoscopic device 100, or the like.

The network unit 230 may acquire an endoscopic video via the endoscopic device 100. Furthermore, when the processor 210 determines a final class for the endoscopic video and generates a control signal for controlling the endoscopic device 100 based on the determined class, the network unit 230 may transmit this control signal to the endoscopic device 100.

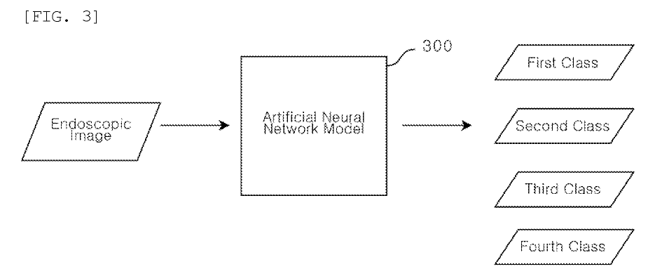

FIG. 3 is an exemplary diagram showing an artificial neural network model according to one embodiment of the present disclosure.

Referring to FIG. 3, an artificial neural network model 300 may be trained to receive an endoscopic image and output a class corresponding to the endoscopic image. In this case, the artificial neural network model 300 may perform learning and inference operations by using the computing device 200 of FIG. 1. In this case, the endoscopic image may refer to each of the frames constituting an endoscopic video.

Labeled endoscopic images may be used as training data for training the artificial neural network model 300. Endoscopic images may each be labeled as one of the first to fourth classes. The first class may be a label for an image corresponding to a case where the air pump of the endoscopic device 100 is determined to require operation. The second class may be a label for an image corresponding to a case where the water pump of the endoscopic device 100 is determined to require operation. The third class is a label for an image corresponding to a case where the suction pump of the endoscopic device 100 is determined to require operation. The fourth class may be a label for an image corresponding to a case where the operation of any one of the air pump, the water pump, and the suction pump is not required.

In the present disclosure, the reason why the fourth class is present is that, when only the first to third classes, in which the pump operates, are present, the pump may operate unnecessarily during an endoscopic procedure, thereby interfering with a medical procedure or posing a safety-related hazard. That is, the presence of the fourth class, which corresponds to a case where the pump does not operate, ensures the safety of the endoscopic procedure.

The first class may include a case where a foreign substance is attached to the lens of the scope 150, resulting in a blurred image, a case where water or a foreign substance is attached to the lens, and a case where an observation area formed by the inner wall of a digestive organ is narrow. The second class may include a case where water or a foreign substance is attached to the lens, requiring the cleaning of the lens, and a case where a foreign substance or blood is present in a lesion region, requiring the cleaning of the affected area. The third class may include a case where secretions (e.g., gastric juice) are present in front of the lens of the scope 150, resulting in a region in contact with the secretions appearing in a different color on an image. The fourth class may include a case where an instrument for an endoscopic procedure is present in an endoscopic image, a case where the endoscopic scope 150 collides with the inner wall of the body, a case where motion blur occurs due to movement of the endoscopic scope 150, and a case where an image is blurred due to an out-of-focus condition. The fourth class may include cases that are difficult to classify as any one of the first to third classes.

In the case where the endoscopic scope 150 collides with the inner wall, a corresponding endoscopic image is classified as the fourth class because the air pump, the water pump, and the suction pump must not operate for safety-related reasons. Furthermore, a case where an endoscopic image is blurred and a case where a foreign substance is attached to the lens may correspond to the first class or the second class. A case where there is the movement of the scope 150 and a case where the lens itself is out of focus may correspond to the third class.

Meanwhile, the computing device 200 may apply various augmentation techniques to endoscopic images in order to obtain training data. In this case, augmentation techniques that do not cause class changes may be used. There may be excluded augmentation techniques based on cropping, which can cause a foreign substance to disappear from an image, or based on blurring, which can generate information that may be confused with a foreign substance.

The artificial neural network model 300 may include a classification model. The classification model may be a lightweight model intended to ensure the real-time performance of endoscopic procedures. For example, the artificial neural network model 300 may include a plurality of convolutional layers, a batch normalization block, an activation function, and a fully-connected layer.

More specifically, for the training of the artificial neural network model 300, the computing device 200 may extract features from endoscopic images included in training data through a convolution operation, batch normalization, and activation function computation. Thereafter, the computing device 200 may output classes and their corresponding probabilities through the fully-connected layer. Based on the correct label for one of the four classes included in the training data, a cross-entropy loss function may be used to calculate the difference between the predicted value of the artificial neural network model 300 and the actual correct label. The gradient of each weight for the value of the loss function may be calculated by using a backpropagation algorithm, and the weights may be updated through gradient descent by using the calculated gradients. There may be performed the process of adjusting the weights of the loss function by repeating the above process.

In this case, the computing device 200 may apply corresponding weights to the first to fourth classes, respectively, during the process of adjusting the weights of the loss function. In this case, the computing device 200 may set the weight for the label of the first or second class to be larger than the weight for the label of the third class. The reason for this is that an image pattern corresponding to the first or second class is much more complex and diverse than that corresponding to the third class, so that focusing on classification as the first or second class can improve the accuracy of learning.

Meanwhile, the artificial neural network model 300 may be trained using, in addition to the supervised learning method described above, semi-supervised learning, which uses training data in which endoscopic images are partially labeled, unsupervised learning, which learns the structures or patterns of endoscopic images themselves without correct labels for training data, and self-supervised learning, which performs learning by using labels automatically generated from endoscopic images.

The trained artificial neural network model 300 may receive input an endoscopic image and output a class corresponding to the endoscopic image. The artificial neural network model 300 may generate a class determination result for each frame by performing inference on an endoscopic image frame.

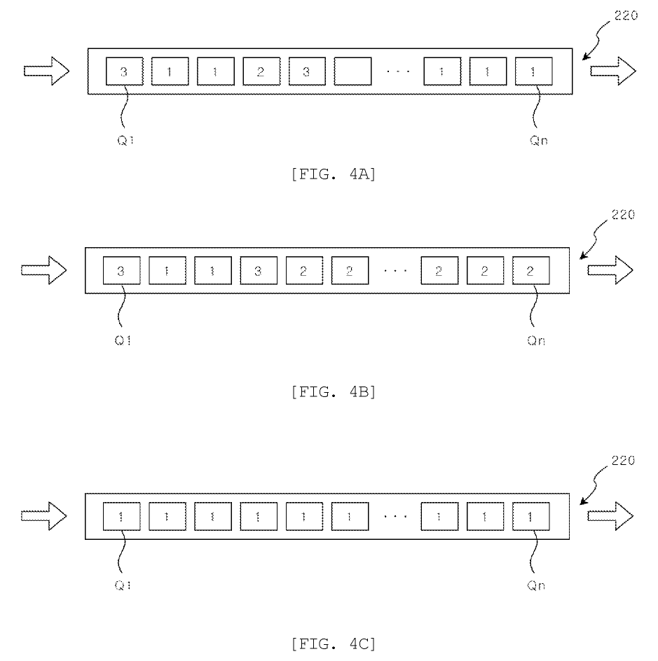

FIGS. 4A, 4B, and 4C are exemplary diagrams showing a method of operating a computing device according to one embodiment of the present disclosure.

Referring to FIGS. 4A, 4B, and 4C, the computing device 200 may determine a final class for operating the pump of the endoscopic device 100 by using frame-wise class results output from the artificial neural network model 300. That is, the computing device 200 may determine a final class by combining a plurality of frame-wise classes. Accordingly, rather than performing an operation based on a frame-wise class result for each frame, the endoscopic device 100 performs an operation based on a cumulative determination result after all determination results for a plurality of frames have been determined. That is, the computing device 200 may make a final determination by taking into consideration the consistency or tendency between a plurality of frame-wise class results.

As an example, the computing device 200 may store frame-wise class results in the memory 220 having a preset size. In this case, the computing device 200 may sequentially store the frame-wise class results according to the order of acquired endoscopic images. The memory 220 may be constructed as part of the memory 220 of FIG. 1.

For example, the memory 220 may have n spaces configured to store n frame-wise class results, and initially, all the n spaces may be filled with the third classes. The memory 220 may be constructed in a queue form, so that information entered first is output first and then information entered later is output sequentially.

Furthermore, the frame-wise class results output from the artificial neural network model 300 may be filled in spaces starting from a first space Q1. Once a frame-wise class result for an n-th frame has been stored, a class result for the earliest input frame may be stored in a space Qn, and a class result for the most recent frame may be stored in the space Q1.

FIGS. 4A, 4B and 4C may show class results stored in the memory 220 at a specific point in time. In this case, after a reference unit of time, e.g., one frame, has elapsed, the frame-wise class results located in n spaces may be shifted to the right by one space. Accordingly, a class result for the most recent frame may be filled in the position of a space Q1.

The computing device 200 may determine a final class based on the continuity and number of per-class determination results in the n spaces of the memory 220.

As an example, when a reference number, e.g., a first number, of spaces, out of the n spaces in the memory 220, are all allocated to the first class as shown in FIG. 4C, the computing device 200 may ultimately determine the first class. In this case, the first number may be larger than a third number to be described below. For example, the first number may be n. The second class may be determined in the same manner as the first class. That is, when a second number of spaces, out of the n spaces in the memory 220, are all allocated to the second class, the computing device 200 may ultimately determine the second class. In this case, the second number may be larger than the third number to be described below.

As an example, in a situation where a reference number, e.g., a third number, of third classes are sequentially arranged in the n spaces of the memory 220, as shown in FIG. 4B, the computing device 200 may ultimately determine the third class. In this case, the third number may be smaller than n.

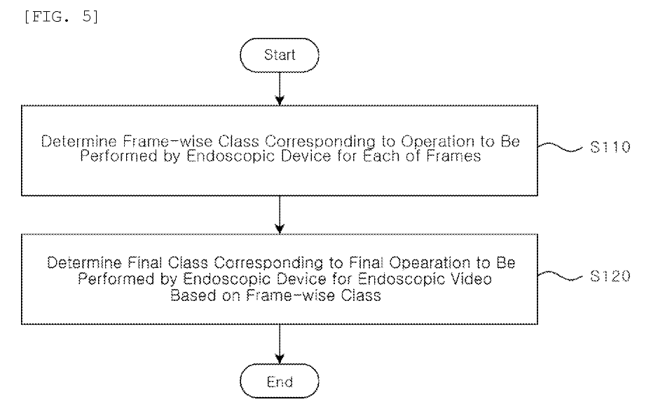

FIG. 5 is a flowchart showing a method by which a computing device controls an endoscopic device according to one embodiment of the present disclosure.

Referring to FIG. 5, the computing device 200 may determine a frame-wise class corresponding to an operation to be performed by the endoscopic device 100 for each frame in an endoscopic video including a plurality of frames in step S110. In this case, the class may include at least one class selected from the group consisting of a first class for operating the air pump of the endoscopic device 100, a second class for operating the water pump of the endoscopic device 100, a third class for operating the suction pump of the endoscopic device 100, and a fourth class for cases other than those of the first to third classes.

The computing device 200 may determine an image containing secretions inside the body, into which the endoscope scope is inserted, to correspond to the third class. The computing device 200 may determine an image, corresponding to a case where the scope of an endoscopic device comes into contact with the inner wall of the body, to correspond to the fourth class. The computing device 200 may determine an image containing noise, generated by the movement of the scope, to correspond to the fourth class. In this case, the noise may include a defocused image or motion blur.

The computing device 200 may determine a final class corresponding to a final operation to be performed by the endoscopic device for the endoscopic video based on the frame-wise classes in step S120. The computing device 200 may determine the final class based on the types and number of frame-wise classes determined for a plurality of frames.

The computing device 200 may determine a class for each of a series of frames, including a first frame, which constitutes the endoscopic video. As an example, the class of the first frame corresponds to one of Q1 to Qn in FIGS. 4A, 4B, and 4C.

In addition, the computing device 200 may determine a final class based on the class determined for at least one previous frame that is earlier than the first frame or the class determined for at least one subsequent frame that is later than the first frame.

In this case, the class determined for at least one previous frame may be stored before the class determined for the first frame, and the class determined for at least one subsequent frame may be stored after the class determined for the first frame. For example, in FIGS. 4A, 4B, and 4C, when a class is determined for the second frame, which is the frame following the first frame, after the class for the first frame has been stored in Q1, the class for the first frame may be stored in Q2, and the class for the second frame may be stored in Q1.

The computing device 200 may store frame-wise class determination results in the memory 220. The computing device 200 may determine a final class based on the types and number of stored classes. More specifically, when a first number of first classes are stored consecutively, the computing device 200 may determine the final class to be the first class. When a second number of second classes are stored consecutively, the computing device 200 may determine the final class to be the second class. Furthermore, when a third number of third classes are stored consecutively, the computing device 200 may determine the final class to be the third class. In this case, the first or second number may be larger than the third number.

The computing device 200 may control the endoscopic device 100 to perform an operation corresponding to the final class. That is, the computing device 200 may control at least one of the air pump, the water pump, or the suction pump to perform an operation corresponding to the final class. The endoscopic device 100 may operate the air pump when the final class is the first class. The endoscopic device 100 may operate the water pump when the final class is the second class. The endoscopic device 100 may operate the suction pump when the final class is the third class.

As an example, the endoscopic device 100 may operate the water pump and then sequentially operate the air pump based on the frame duration of a frame-wise class. That is, based on the intensity of cleaning required, the endoscopic device 100 may operate the water pump for a preset period of time, terminate the operation, and then operate the air pump for a preset period of time.

For example, when the second class is determined for 30 frames, the endoscopic device 100 may operate the water pump for a period of time corresponding to 30 frames, and then operate the air pump for a specific period of time regardless of the frame-wise class results.

Even when the endoscopic device 100 operates the water pump based on the second class for a specific number of frames (e.g., 20 frames), it is determined that the operation of the water pump is still necessary, and thus it is determined that high-intensity cleaning is necessary. Accordingly, the air pump may be additionally operated to enhance cleaning power.

Class results are generated on a frame-wise basis. Accordingly, when the endoscopic device 100 is controlled on a frame-wise basis, an operation may change on a frame-wise basis. The present disclosure controls the endoscopic device 100 by taking into consideration the continuity of a plurality of frame-wise classes for an endoscopic video to ensure stable operation. A case of controlling the endoscopic device 100 based solely on frame-wise classes will be described as an example. As an example, as for an endoscopic video composed of three frames, when the classes of the respective frames are the third class, the fourth class, and the third class, the suction pump needs to be operated for one frame, then stopped for another frame, and then operated for another frame. In an actual endoscopic procedure environment, the endoscopic image varies significantly, and thus the determination of the artificial neural network model 300 may rapidly change on a frame-wise basis. Therefore, in the present disclosure, for consistent and robust operation, the pump of the endoscopic device 100 may be controlled when a series of class determination results corresponding to a predetermined number are successively output.

The above description of the present disclosure is intended for illustrative purposes only, and those skilled in the art will readily appreciate that modifications into other specific forms may be made without altering the technical spirit or essential features of the present disclosure. Therefore, the embodiments described above should be understood as illustrative and not restrictive in all respects. For example, components described as single may be implemented in a distributed form, and similarly, components described as distributed may be implemented in a combined form.

The scope of the present disclosure is defined by the attached claims rather than the above detailed description, and all changes or modifications derived from the meanings and scope of the claims and their equivalent concepts should be interpreted as being encompassed in the scope of the present disclosure.

What is claimed is:

1. A method of analyzing endoscopic video, the method being performed by a computing device including at least one processor, the method comprising:
determining a frame-wise class corresponding to an operation to be performed by an endoscopic device for each frame in an endoscopic video including a plurality of frames; and
determining a final class corresponding to a final operation to be performed by the endoscopic device for the endoscopic video based on the frame-wise class;
wherein the class comprises at least one class selected from the group consisting of:
a first class for operating an air pump of the endoscopic device;
a second class for operating a water pump of the endoscopic device;
a third class for operating a suction pump of the endoscopic device; and
a fourth class corresponding to cases other than cases of the first to third classes.

2. The method of claim 1, wherein determining the final class comprises determining the final class based on a class determined for at least one previous frame earlier than a first frame or a class determined for at least one subsequent frame later than the first frame.

3. The method of claim 2, wherein determining the final class comprises determining the final class based on a type and number of frame-wise classes determined for the plurality of frames.

4. The method of claim 3, wherein determining the frame-wise class comprises storing the class determined for the at least one previous frame before the class determined for the first frame, and storing the class determined for the at least one subsequent frame after the class determined for the first frame.

5. The method of claim 4, wherein determining the final class comprises:
when the first class is stored successively for a first number of times, determining the final class to be the first class;
when the second class is stored successively for a second number of times, determining the final class to be the second class; and
when the third class is stored successively for a third number of times, determining the final class to be the third class.

6. The method of claim 5, wherein the first number or the second number is larger than the third number.

7. The method of claim 1, further comprising controlling at least one of the air pump, the water pump, or the suction pump to perform an operation corresponding to the final class.

8. The method of claim 1, wherein determining the frame-wise class comprises determining an image containing secretions inside a body, into which a scope of the endoscopic device is inserted, to be the third class.

9. The method of claim 1, wherein determining the frame-wise class comprises determining an image, corresponding to a case where a scope of the endoscopic device comes into contact with an inner wall of a body, to be the fourth class.

10. The method of claim 1, wherein determining the frame-wise class comprises determining an image, containing noise caused due to movement of a scope, to be the fourth class.

11. The method of claim 10, wherein the noise comprises a defocused image or motion blur.

12. A computing device for analyzing endoscopic video, the computing device comprising:
memory configured to store an endoscopic video including a plurality of frames; and
a processor configured to determine a frame-wise class corresponding to an operation to be performed by an endoscopic device for each of the frames and determine a final class corresponding to a final operation to be performed by the endoscopic device for the endoscopic video based on the frame-wise class;
wherein the class comprises at least one class selected from the group consisting of:
a first class for operating an air pump of the endoscopic device;
a second class for operating a water pump of the endoscopic device;
a third class for operating a suction pump of the endoscopic device; and
a fourth class corresponding to cases other than cases of the first to third classes.

13. A computer program stored in a computer-readable storage medium, the computer program causing operations for analyzing endoscopic video to be performed when executed by at least one processor, wherein the operations comprise operations of:
determining a frame-wise class corresponding to an operation to be performed by an endoscopic device for each frame in an endoscopic video including a plurality of frames; and
determining a final class corresponding to a final operation to be performed by the endoscopic device for the endoscopic video based on the frame-wise class;
wherein the class comprises at least one class selected from the group consisting of:
a first class for operating an air pump of the endoscopic device;
a second class for operating a water pump of the endoscopic device;
a third class for operating a suction pump of the endoscopic device; and
a fourth class corresponding to cases other than cases of the first to third classes.
